(19) United States
(12) Patent Application Publication
(10) Pub. No.: US 2018/0360396 A1
(43) Pub. Date: Dec. 20, 2018

(54) IMAGING APPARATUS AND POSITIONING APPARATUS

(71) Applicant: Siemens Healthcare GmbH, Erlangen (DE)

(72) Inventors: De He Weng, Shenzhen (CN); Xu Yan, Shenzhen (CN)

(73) Assignee: Siemens Healthcare GmbH, Erlangen (DE)

(21) Appl. No.: 16/124,660

(22) Filed: Sep. 7, 2018

Related U.S. Application Data
(62) Division of application No. 14/854,434, filed on Sep. 15, 2015.

(30) Foreign Application Priority Data
Sep. 15, 2014 (CN) 201410469422.3

(51) Int. Cl. A61B 6/04; G01S 5/30; A61B 5/055; A61B 6/03

(52) U.S. Cl. CPC A61B 6/0492; G01S 5/30; A61B 5/0555; A61B 6/032; A61B 2019/5429; A61B 2019/524; A61B 2019/5236; A61B 2019/5445; A61B 2019/5479

(57) ABSTRACT

An imaging apparatus and a positioning apparatus that include an imaging unit for imaging a detected part of a body to be detected; and a distance positioning unit having at least two signal positioning devices, i.e., a signal generator and a signal detector, wherein one signal positioning device is arranged at the detected part, the other signal positioning device is arranged at a position where a predetermined position relationship exists relative to the imaging unit. The position of the detected part in at least one direction in a three-dimensional space relative to the imaging unit is obtained according to a measured distance between the two signal positioning devices. The imaging apparatus and the positioning apparatus reduce pre-positioning imaging sequence workload, and the risk of radiation on the body to be detected is avoided.

### RELATED APPLICATION

The present application is a divisional application of Ser. No. 14/854,434, filed on Sep. 15, 2015, the contents of which are incorporated herein by reference.

### BACKGROUND OF THE INVENTION

### Field of the Invention

The present invention relates to an imaging apparatus and a positioning apparatus useable therein.

### Description of the Prior Art

For current health and disease examinations, the use of magnetic resonance (MR) imaging or computed tomography (CT) imaging is increasing. During such imaging and scanning by a large apparatus, the body to be examined is usually required to lie, and be positioned, in a target region. The positioning target is a predetermined scanning center during imaging. Conventionally, a positioning procedure is always implemented using a laser marker or by a series of position detecting sensors in a longitudinal direction.

Such conventional positioning methods can determine only a transverse section of the detected body by defining a certain length in the longitudinal direction. However, MR imaging and CT imaging always require a clear target of a certain organ or region of the human body. To this end, not only the positioning of a transverse section, but also the positioning of a sagittal section and a coronal section are required.

FIG. 12 illustrates a conventional positioning procedure using a laser marker. The use and workflow of an MR system will be explained as an example. A body 1 to be detected lies on the top of an examining table 2, and a marker or coil is provided on the body 1 to be detected. An operator makes a laser 3 project vertically downwardly by operating the laser 3 on an imaging unit 4 so as to form a marking line on the body to be detected, or on the coil therebelow, such that the marking line is aligned with a marker provided on the body 1 to be detected. Since the laser marker is installed at a predetermined position on the imaging unit and the distance a* from the center of the imaging unit is known, the distance from a marking point on the body to be detected to the center of the imaging unit can be calculated. Then the operator moves the body to be detected to an imaging center A according to this distance, and starts a pre-positioning imaging sequence. With images from the pre-positioning imaging sequence, the operator needs to redefine an exact imaging region in three directions, the three directions respectively constituting the sagittal section, the coronal section and the transverse section.

In the method described above, the MR system allows only direct positioning in the longitudinal direction, i.e. positioning of the transverse section, and does not allow direct positioning of the sagittal section and the coronal section. Therefore, even if an operator who has knowledge of imaging is clearly aware of which part needs to be scanned, this traditional positioning method cannot transform the operator's intention directly into imaging, and the operator needs to run a special pre-positioning imaging sequence to redefine the transverse section, the sagittal section and the coronal section, so as to determine the final target imaging position, and after that a real imaging examination can be carried out. This will require the operator to perform extra work in order to identify the correct target position. In this positioning method, the work of re-adjusting is inevitable.

In addition, there is a potential safety risk with the laser marker, which is harmful to the eyes of the body to be detected due to the radiation. This requires additional safety measures and means, and makes the system more complex.

Another positioning apparatus is provided in CN 202288286 U. An active detector is used instead of a laser marker. The active detector can detect a target object passing directly therebelow by using a reflector provided on the body to be detected and local coils etc. when the body to be detected is being moved into the imaging unit. In principle, this method also follows the same distance calculation as that with the positioning apparatus described above, which improves the workflow by omitting the manually marking step.

CN 201591629 U illustrates another positioning apparatus, which is provided with a series of sensors or switches in a longitudinal direction along an examining table, and which then detects a target transverse section by triggering a local coil or orientation block.

To date, all MR and CT systems, including those in the above-mentioned patent documents and in the prior art, have used the same positioning method of defining a transverse section in a longitudinal direction of the body to be detected. However, as described above, all these methods are only able to achieve longitudinal positioning, and the operator still requires a pre-positioning imaging sequence to obtain a pre-positioning image for determining the final target imaging position.

### SUMMARY OF THE INVENTION

An object of the present invention is to provide an imaging device and a positioning apparatus that are capable of reducing pre-positioning imaging sequence workload and avoiding the risk of radiation on the body to be detected.

The present invention in one aspect thereof provides an imaging device, having an imaging unit (scanner) for imaging an examined part of a body to be examined, and a distance positioning unit that includes at least two signal positioning devices, i.e., a signal generator and a signal detector, wherein one signal positioning device is arranged at the detected part, the other signal positioning device is arranged at a position where a predetermined position relationship exists relative to the imaging unit. The position of the detected part in at least one direction in space relative to the imaging unit is obtained according to a measured distance between the two signal positioning devices.

In the imaging apparatus of the present invention, which operates according to the inventive method, the target imaging on the patient's body can be recognized in one step and the body can be automatically moved to the isocenter, thus reducing the pre-positioning imaging sequence workload, improving the workflow efficiency, and meanwhile avoiding the errors due to manual positioning by an operator of the imaging device, and thus being particularly effective for an open imaging unit in a current imaging unit, such as a permanent magnet-type magnetic resonance imaging device. Furthermore, in the present invention, the wording “position in at least one direction in space” means a position of distance along any one of an X axis, a Y axis or a Z axis in a spatial coordinate system.

According to the distance/angle positioning unit in the imaging apparatus of the present invention, the operator of the imaging apparatus can use his knowledge of anatomy and iconography to directly define a center to be imaged in a single operation and at any location on the patient's body, this will help the operator to create the imaging of the patient closer to the diagnostic requirements. The imaging apparatus according to the present invention will save significant preparation time for the operator, thereby increasing the operating efficiency of the imaging device, and improving the health examination capability of a hospital/clinic.

In addition, the imaging apparatus according to the present invention can transmit a detection signal from the signal generator to a signal receiver provided at the imaging unit. Since the signal radiation path is higher than the patient, there is no risk of radiation on the patient, and thus additional protective apparatuses are unnecessary and the associated costs can be reduced.

In addition, the imaging apparatus according to the present invention allows positioning in a three-dimensional space, and with the development of imaging devices in the future, the function of positioning in a three-dimensional space will be more helpful. By means of positioning in a three-dimensional space, a coordinate system can be established based on the operator's intention, which will help to further improve the image reconstruction, quality as well as speed.

Furthermore, the imaging apparatus of the present invention preferably further has an angle positioning unit that includes at least one auxiliary signal positioning device, wherein the auxiliary signal positioning device is arranged at the detected part, and is formed by one of the signal generator and the signal detector. An angle of the detected part in at least one direction in space relative to the imaging unit is obtained according to the auxiliary signal positioning device and a distance difference between the one signal positioning device and the other signal positioning device. In the present invention, the term “angle in at least one direction in space” means a rotation angle around the X-axis, Y-axis or Z-axis in the spatial coordinate system.

Furthermore, the imaging apparatus of the present invention preferably has three or more other signal positioning devices, with a predetermined position relationship existing between the other signal positioning devices, and the position of the detected part in space relative to the imaging unit is determined according to a distance between the one signal positioning device and each of the other signal positioning devices. In the present invention, the expression “position in space” means a position of distance along the X-axis, Y-axis and Z-axis in the spatial coordinate system.

Furthermore, in the imaging apparatus of the present invention, the angle positioning unit preferably further has multiple angle sensors arranged at the detected part.

Furthermore, in the imaging apparatus of the present invention, the signal generator is preferably any of an ultrasonic signal generator, an infrared signal generator, and a laser signal generator.

Furthermore, the imaging apparatus of the present invention preferably further has a control computer, and the control computer controls the imaging unit to image the detected part according to the position determined by the distance positioning unit.

Furthermore, in the imaging apparatus of the present invention, the imaging apparatus preferably further has a control computer, and the control computer controls the imaging unit to image the detected part according to the position determined by the distance positioning unit and the angle determined by the angle positioning unit.

Furthermore, the present invention in a further aspect thereof provides a positioning apparatus, having a distance positioning unit that includes at least two signal positioning devices, i.e., a signal generator and a signal detector, wherein one signal positioning device is arranged at a detected part, the other signal positioning device is arranged at a position in space where a predetermined position relationship exists, and a position of the detected part in at least one direction in space is obtained according to a measured distance between the two signal positioning devices. The positioning apparatus further has an angle positioning unit that includes at least one auxiliary signal positioning device, wherein the auxiliary signal positioning device is arranged at the detected part, and is formed by one of the signal generator and the signal detector. An angle of the detected part in at least one direction in space is obtained according to the auxiliary signal positioning device and a distance difference between the one signal positioning device and the other signal positioning device.

The positioning apparatus according to the present invention can provide a direct and accurate position and angle positioning in space.

Furthermore, in the positioning apparatus of the present invention, the angle positioning unit preferably further comprises a plurality of angle sensors arranged at the detected part.

### BRIEF DESCRIPTION OF THE DRAWINGS

FIG. 1 is a schematic illustration of a distance positioning unit of an imaging device according to a first embodiment of the present invention.

FIG. 2 is a diagram that explains the positioning principle of the distance positioning unit in the present invention.

FIG. 3 is a diagram that explains the positioning of a transverse section using the distance positioning unit of the first embodiment.

FIG. 4 is a diagram that explains the positioning of a sagittal section using the distance positioning unit of the first embodiment.

FIG. 5 is a diagram that explains the positioning of an original coronal section using the distance positioning unit of the first embodiment.

FIG. 6 is a diagram that explains the determination of a final coronal section.

FIG. 7 is a structural diagram representing an angle positioning unit of a second embodiment.

FIG. 8 is a diagram that explains the angle positioning in one direction using the angle positioning unit of the second embodiment.

FIG. 9 is a diagram that explains the angle positioning in one direction using the angle positioning unit of the second embodiment.

FIG. 10 is a diagram that explains the angle positioning in one direction using the angle positioning unit of the second embodiment.

FIG. 11 is a diagram that explains a method of two-dimensional positioning using a distance positioning unit of the present invention.

FIG. 12 is a diagram that explains a prior art positioning apparatus.

### DESCRIPTION OF THE PREFERRED EMBODIMENTS

In the figures, reference characters are as follows:

1 body to be detected; 2 examining bed; 3 detected part; 4 imaging unit; 5 indicator; 6, 7, 8 signal detector; 9 signal generator; 10 handle; 11, 12 angle detector; and 13 auxiliary signal generator.

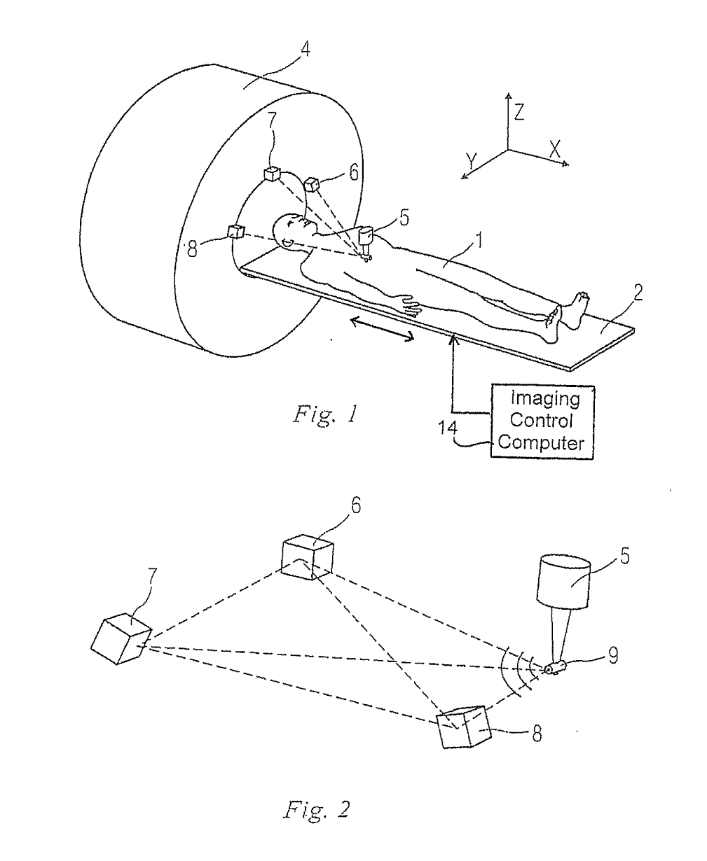

FIG. 1 schematically illustrates an imaging apparatus in a first embodiment of the present invention. In FIG. 1, a magnetic resonance (MR) imaging device is explained as an example. The imaging device has an examining bed 2 carrying a body 1; a distance positioning unit for positioning a part of the body 1 that is to be imaged; and an imaging unit (magnet) 4 (commonly called a scanner) having an imaging region for imaging the part of the body 1 that is to be imaged. In the present embodiment, the body 1 lies on the examining bed 2, and a distance of the part of the body 1 to be imaged relative to the imaging unit 4 is determined by the distance positioning unit, and then the examining bed 2 is operated by an imaging control computer 14 so as to convey (as indicated by the double arrow) the body 1 into the imaging unit 4, and align the part to be imaged with the imaging region of the imaging unit 4 according to the distance determined by the distance positioning unit, such that the imaging unit 4 obtains image data from the part to be imaged.

The distance positioning unit in the present embodiment is a spatial distance positioning unit used for detecting a spatial distance. The spatial distance positioning unit adopts a direct measurement method and comprises an indicator 5 as a positioning signal transmitting system and signal detectors 6, 7, 8 as a positioning signal receiving system. The indicator 5 is a hand-held device, which has a signal generator 9 capable of transmitting a positioning signal. The signal detectors 6, 7, 8 are installed on the imaging unit 4 and capable of receiving the positioning signal transmitted by the signal generator 9. The three signal detectors 6, 7, 8 are of the same type, but are installed at different positions, and the three signal detectors 6, 7, 8 have a determined position relationship relative to the isocenter of the imaging unit. When the body 1 to be detected lies on the examining table 2, an operator can use an indicator 5 to mark the target at any position on the body to be detected depending on the organ required to be imaged.

In the present invention, the signal generator 9 and the signal detectors 6, 7, 8 in the distance positioning unit are preferably ultrasonic elements. This is because ultrasonic wave is one of the radiations which are currently confirmed to be safe in the current health care field. Moreover, an ultrasonic sensor can be used as an actuator, or as a detector. When powered on, the ultrasonic sensor converts electrical energy into a mechanical vibration to emit ultrasonic waves. On the contrary, if the sensor is arranged in a receiving circuit, then it can convert the received ultrasonic signal into an electrical signal. In addition, the detection and analysis of the ultrasonic signal are simple and easy, so that the costs can be easily controlled.

In addition, the ultrasonic radiation does not follow a single line. The ultrasonic radiation can maintain an effective amplitude within a beam angle. In the present embodiment, an ultrasonic generator having a beam angle of 60 degrees is selected. The same applies to the three detectors. It is contemplated that the three detectors are all placed higher than the body of the patient. The large beam angle of both the actuator and the detector will ensure an effective detection over the entire region of the body, even if the actuator is placed around the patient's head/foot and very close to the imaging unit. Thus, a distance detection can be realized by only a single actuation source.

FIG. 2 illustrates the positioning principle and basic components of the distance positioning unit. The indicator 5 comprises an operator-held handle 10 and a signal generator 9. The handle 10 has an elliptical shape in the normal direction of a palm or a similar shape having smaller dimensions. With this shape, the operator makes it possible to emit a radiation signal from the indicator 5 to the imaging unit 4 in a substantially normal direction. In addition, the bottom of the handle 10 is of an inverted conical configuration, which can indicate the direction clearly during positioning. The signal generator 9 is mounted on the bottom of a needle-like structure on the handle, and the signal generator 9 transmits ultrasonic signals at a determined pulse interval. The ultrasonic signal is attenuated during propagation. The signal receiving system can use the pulse interval of the ultrasonic signals to identify the stage of measurement start, measurement ongoing and measurement end, and in this way, influence from coupling noise can be avoided.

The signal detectors 6, 7, 8 as the positioning signal receiving system are arranged at a position where a predetermined position relationship exists relative to the isocenter of the imaging unit, and the position relationship between the individual signal detectors 6, 7, 8 is also fixed; in the embodiments which follow, each signal detector is arranged at the imaging unit 4 and has a predetermined position relationship relative to the isocenter of the imaging region of the imaging unit 4, and all the signal detectors are configured to be located at the three vertexes of an equilateral triangle, but are not limited thereto, as long as they have a determined and known position relationship. After ultrasonic signals transmitted from the signal generator 9 are detected, since the distances from the signal generator 9 to the signal detectors 6, 7, 8 are different, the signal detectors 6, 7, 8 receive ultrasonic signals at different levels, and the signal detectors 6, 7, 8 compare the received ultrasonic signals with a reference amplitude while taking into account compensation factors at different temperatures, in order to calculate the signal attenuation, and to obtain the distances from the signal generator 9 to the signal detectors 6, 7, 8 according to the signal attenuation. Thus, a tetrahedron is formed in the spatial positioning system with the signal generator 9 and the signal detectors 6, 7, 8 as vertexes, and the length of each side of the tetrahedron is known.

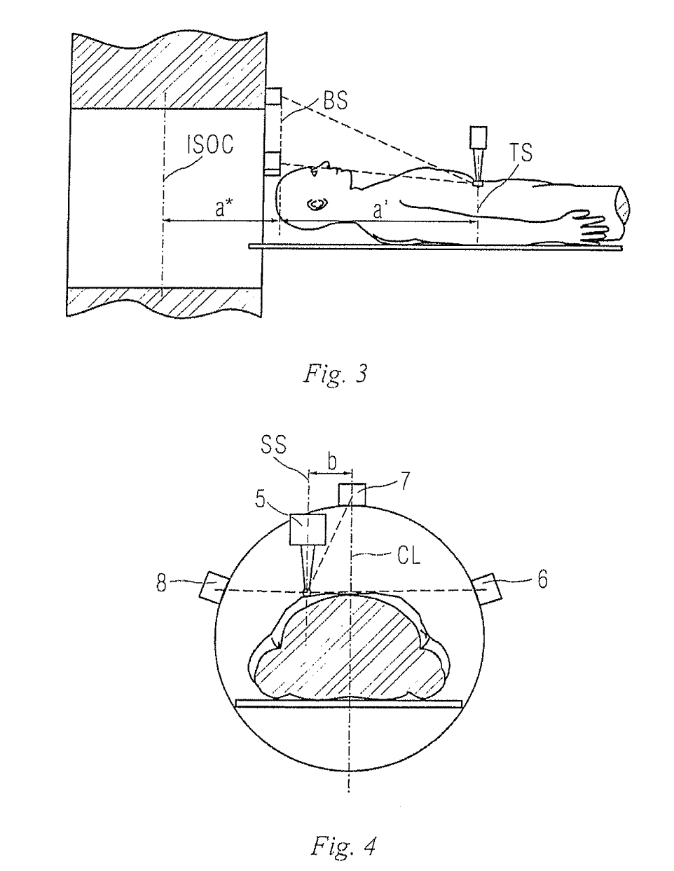

The positioning of the detected part using the distance positioning unit will be explained below particularly with reference to FIGS. 3-5.

The operator firstly places the indicator 5 at the part to be scanned of the body to be detected. Then the indicator 5 is enabled to work, and when the indicator 5 starts working, the signal generator 9 transmits ultrasonic signals to the signal detectors 6, 7, 8 arranged on the imaging unit 4; since the signal detectors are arranged on the imaging device at different positions, they will each perform an analysis to obtain the distance between the signal generator 9 and the respective one of the signal detectors 6, 7, 8. In addition, a base surface where the signal detectors 6, 7, 8 are located is aligned with the front part of the imaging unit, and as described above, the distance from the base surface to the isocenter of the imaging unit is fixed. Here, the marking position is not only the position information in the longitudinal direction (transverse section), but also the position information in the sagittal and coronal directions.

FIG. 3 illustrates the positioning of the transverse section TS. Utilizing the tetrahedron each side of which is known, and according to geometric calculations, the height from the position of the vertex where the signal generator 9 is located relative to the base surface BS can be calculated to be equal to the direct distance “a′” from the target position on the patient to the front part of the imaging unit. Moreover, the distance “a*” between the front part of the imaging unit and the isocenter ISOC of the imaging unit is a fixed parameter obtained based on the mechanical assembly of the system. It can be seen that the distance from the target transverse section to the isocenter of the imaging unit is a=a′+a*.

FIG. 4 illustrates the positioning of a sagittal section SS. FIG. 6 is a sectional view taken along the transverse section. The sagittal section SS is perpendicular to the transverse section and oriented in the longitudinal direction. For example, when it is to scan the patient's right chest region, the indicator 5 is placed at the part to be detected, constituting a vertex of the tetrahedron in the positioning system. In the present embodiment, the center line of the equilateral triangle formed by the detectors 6, 7, 8 and located on the base surface BS coincides with the center line CL at the isocenter of the imaging unit. Thus, the distance b from the sagittal section of the target region on the patient's body to the isocenter of the imaging unit can be obtained by geometric calculations, i.e., the distance between the sagittal section SS in which the indicator 5 is located and the center line CL passing through the isocenter.

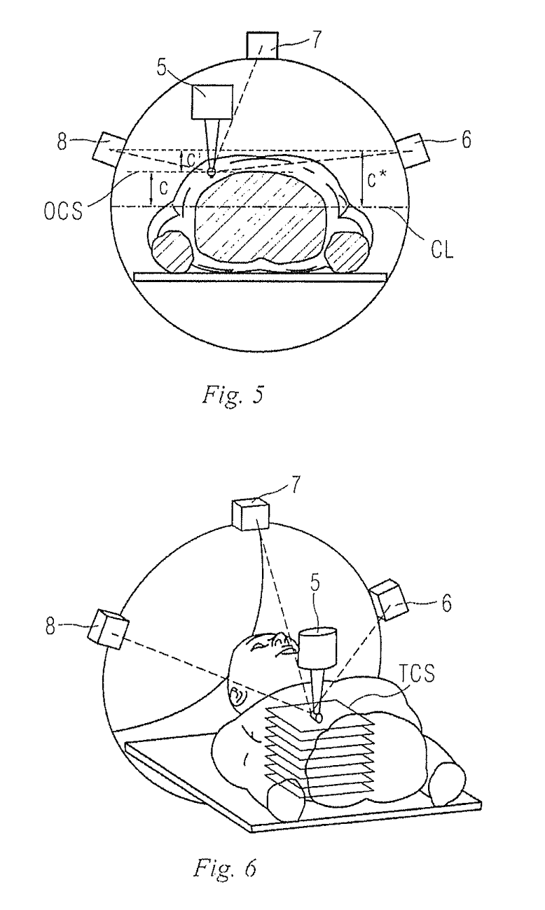

FIG. 5 illustrates the positioning of an original coronal section CS. In FIG. 5, the sectional view taken along the transverse section is still used as an example to illustrate the positioning of the coronal section. The positioning of the coronal section is slightly different from the other two sectional directions, and the defining of the coronal section comprises measurement of the original coronal section OCS and the determination of a target coronal section TCS. Firstly illustrated is the measurement of the original coronal section OCS. The indicator 5 is placed at the part to be detected on the patient's body to constitute a vertex of the tetrahedron for positioning, and the distance c from the original coronal section OCS to the center line CL passing through the isocenter is the distance between a plane passing through the signal detectors 6, 8 and parallel to the original coronal section and the center line CL of the imaging unit “c*” plus/minus the distance “c′” between the plane and the vertex where the indicator 5 is located, and can be expressed in the form of an equation as c=c*±c′. c* is determined once each of the signal detectors is assembled, and c′ can be derived by space geometry calculations from the distance between the signal generator 9 and the signal detectors 6, 8 that is measured by the distance positioning unit. Thus, the spatial position of the original coronal section OCS can be defined.

According to the original coronal section OCS measured as above, the thickness of the target region on the patient can be calculated by adding the obtained distance c from the original coronal section OCS to the center line CL to the fixed distance from the center line to the top of the examining table. FIG. 6 is a diagram for explaining the determination of a target coronal section TCS. As shown in FIG. 6, the operator obtains a series of coronal sections at the position by scanning according to the original coronal sectional OCS, and then the imaging device can select the most appropriate coronal section from the series of coronal sections based on pre-stored iconography and human body data, thereby positioning the final target coronal sectional TCS.

At this point, the imaging center of the detected part can be determined by the transverse section TS, the sagittal section SS and the target coronal sectional TCS which are determined by the distance positioning unit, namely the intersection of the three planes. Thus, the operator can directly and quickly achieve the positioning of the detected part, with no or reduced pre-positioning imaging sequence workload like before.

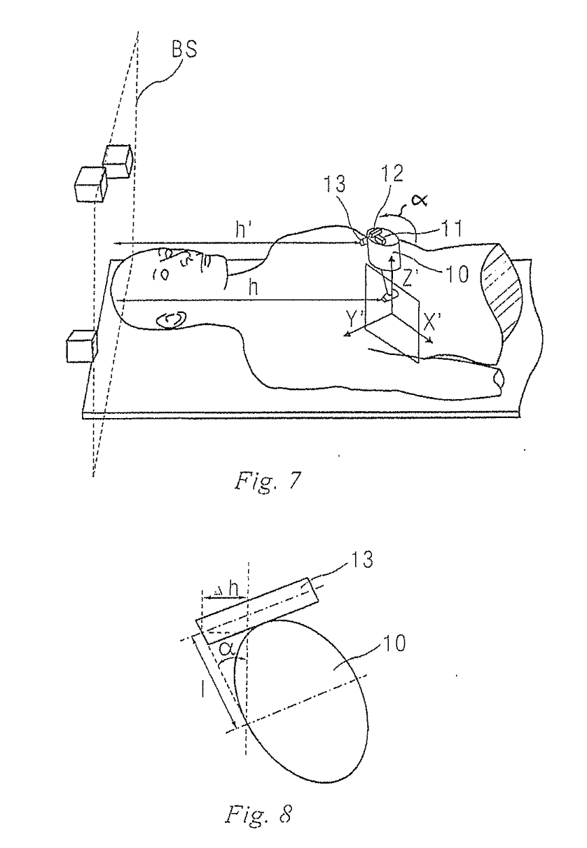

As noted above, the imaging device has an imaging control computer 14, and after the detected part is marked by the indicator 5 and its position is detected by the signal detectors 6, 7 8 of the distance positioning unit, the imaging control computer controls the imaging of the detected part by operating a bed movement controller so as to move the bed 2 according to the distance determined by the distance positioning unit. In everyday diagnostic health examination, in the imaging of the imaging unit, not only the image in the vertical direction is provided, but also rotation or overturning by a certain angle is done sometimes for easier diagnosis. In this case, on the basis of the first embodiment, the present embodiment further provides an angle positioning unit capable of detecting an angle by which the indicator 5 rotates and overturns in space. The structure and angle detection of the angle positioning unit of the present embodiment will be explained below with reference to FIG. 7. FIG. 7 shows a structural view of the angle positioning unit. Except for the structure of the indicator 5 which is different from the first embodiment, the remaining configuration of the angle positioning unit is the same as that of the first embodiment.

As shown in FIG. 7, in the indicator 5 of the present embodiment, in addition to the signal generator 9, an auxiliary signal generator 13 is further provided on a side face of the handle 10, for calculating the rotation angle of the indicator 5 around the Z axis. Furthermore, the handle 10 is further provided with two angle detectors 11, 12 on the top face. The angle detectors 11, 12 may be gravity angle sensors for detecting the rotation angles of the indicator 5 around the X axis and the Y axis.

Furthermore, the handle 10 is internally provided with a power supply far supplying power to the signal generator 9, the auxiliary signal generator 13, the angle detectors 11, 12 and the signal circuit. Furthermore, the indicator 5 further has a wireless communication subsystem for establishing a communication between the handle 10 and a receiving circuit. The signal circuit in the handle can transmit the detected angle information to the receiving system via a wireless signal.

The angle detecting method of the present embodiment will be explained below.

Reference is made to FIG. 7 which illustrates the detection of the rotation angle around the Z axis by the auxiliary signal generator 13. If the indicator 5 rotates around the Z axis by an angle α, first of all by using the method shown in FIG. 3, the projected distance “h” between the signal generator 9 and the base surface BS formed by the signal detectors 6, 7, 8 is obtained by means of the measurement by the indicator 5 and calculation; the auxiliary signal generator 13 also transmits ultrasonic waves, and the projected distance “h′” between the auxiliary signal generator 13 and the base surface BS formed by the signal detectors 6, 7, 8 is obtained by the same method. Then, as shown in FIG. 8, since a difference “Δh” exists between “h” and “h′” and moreover the distance “I” from the auxiliary signal generator 13 to the center of the signal generator 9 is a fixed parameter related to the structure of the handle 10, the rotation angle “α” can be calculated from the detected Δh according to a trigonometric function α=arcsin (Δh/I), thereby obtaining the rotation angle around the Z axis.

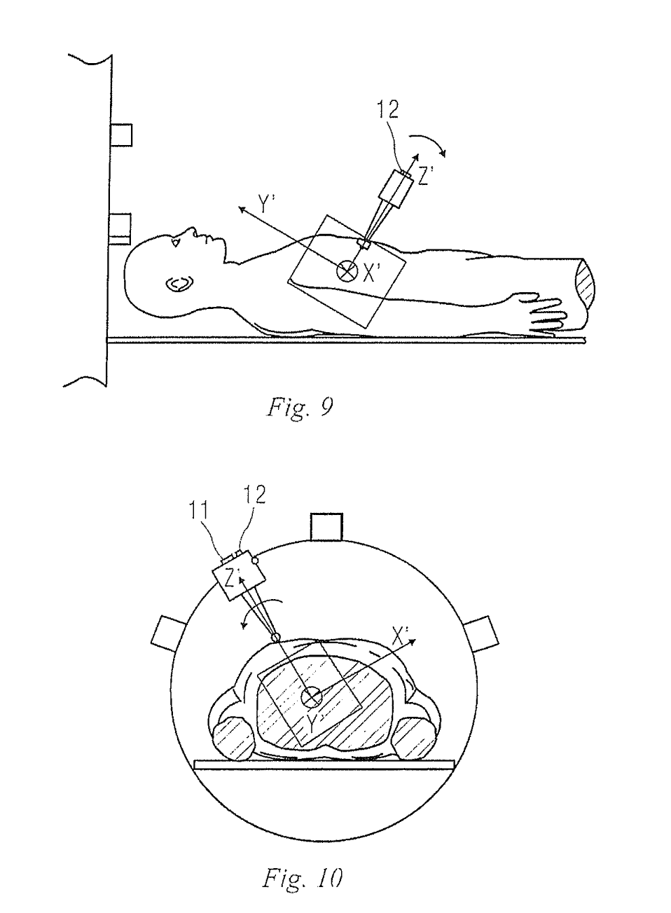

The rotation angles around the X axis and the Y axis can be obtained by direct measurements from the two angle detectors 11, 12. As shown in FIG. 9, the hand-held handle 10 makes the indicator 5 overturn around the X axis by an angle, and the angle detector 12 directly measures this angle and transmits the measured angle information to the receiving system. Similarly, as shown in FIG. 10, the auxiliary signal generator 13 makes the indicator 5 rotate around the Y axis by an angle, and the angle detector 11 directly measures this angle and transmits the measured angle information to the receiving system.

As mentioned above, with the present embodiment, the angle positioning unit can calculate or measure the angle by which the indicator 5 rotates or overturns in space, so that the indicator 5 can be used to indicate the angle of the detected part of the body to be detected in the spatial coordinate system.

The imaging device further has an imaging control computer (not shown in the figures), and after the detected part is marked by the indicator 5 and is determined in terms of distance and angle by the distance positioning unit and/or the angle positioning unit, the imaging control computer controls the imaging of the detected part according to the distance determined by the distance positioning unit and/or the angle determined by the angle positioning unit.

In the first and second embodiments described above, for an illustrative but nonlimiting purpose, three signal detectors 6, 7, 8 are provided at the imaging unit, one signal generator 9 is provided at the detected part of the body to be detected; for example, in the case of multiple detected parts, unlike the above embodiments, it is also possible to provide a plurality of, such as three, signal generators 9, and to provide the three signal generators 9 at the imaging unit in a predetermined position relationship, and to provide an equal number of signal detectors to the number of the detected parts at the detected parts. For example, the three signal generators provided on the imaging unit transmit ultrasonic signals successively, and the ultrasonic signals are received by a plurality of signal detectors, so that the positions of the plurality of signal detectors relative to the signal generators are determined, and in turn the positions of the multiple detected parts relative to the imaging unit are determined; this method is particularly applicable in the case that a plurality of detected parts need to be positioned at the same time.

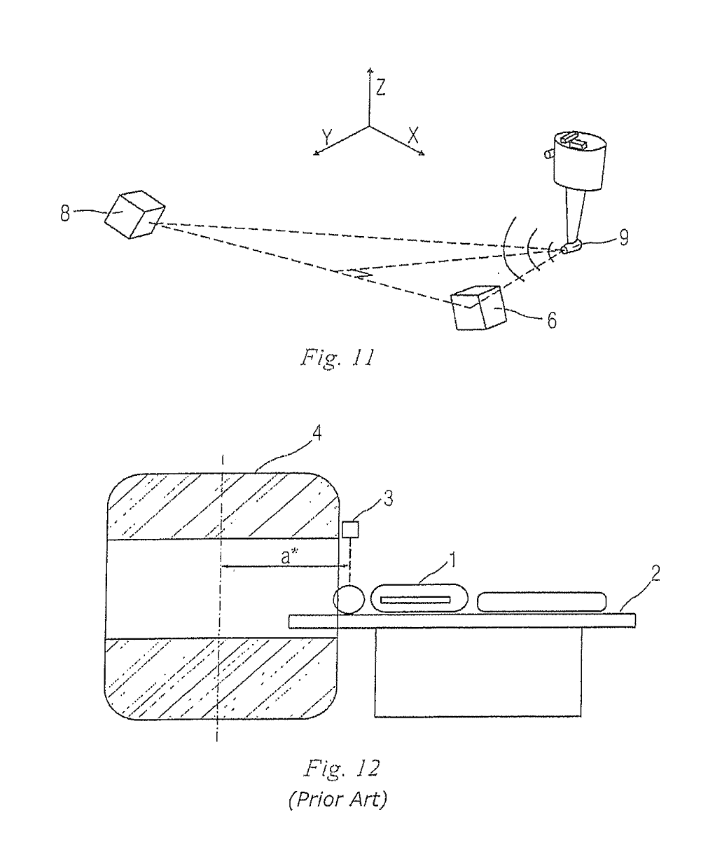

In the embodiments described above, the positioning of a detected part in a three-dimensional space is described as an example; however, the present invention can also be applied to the positioning in two directions or one direction. For example, as shown in FIG. 11, in this embodiment, compared with the first embodiment, if the middle signal detector 7 is omitted, then the two signal detectors 6, 8 and the signal transmitter 9 can constitute a triangle in a plane. The height in the horizontal plane or the projected height will be the distance from the target position to the front part of the imaging unit, namely the position in the X direction in the spatial coordinate system. Meanwhile, the position of the signal emitter 9 in the Y direction can also be identified.

Similarly, in the embodiments described above, a method for measuring the angles around the X, Y, Z axis angles respectively is illustrated; however, the angle(s) around only one or two of the axes may be measured according to actual requirements.

Furthermore, in the embodiments described above, the focus is on the distance and angle detection in a single direction. In practical use, the operator may define the image center in multiple directions and at multiple angles as claimed in his intention, which will bring about great convenience.

Furthermore, in the embodiments described above, the positioning signal is selected to be an ultrasonic signal, but can also be selected to be infrared or laser, that is to say the signal generator 9 may be any one of an ultrasonic signal generator, a laser signal generator and an infrared signal generator.

In the embodiments described above, the magnetic resonance imaging apparatus is described as an example; however, the technical solution of the present invention can also be applied to other imaging devices in which the detected part needs to be positioned, such as a CT apparatus.

Although modifications and changes may be suggested by those skilled in the art, it is the intention of the inventors to embody within the patent warranted hereon all changes and modifications as reasonably and properly come within the scope of their contribution to the art.

We claim as our invention:

1. A medical imaging apparatus comprising:
a data acquisition scanner operable to acquire image data from a subject, said data acquisition scanner having a subject-receiving opening therein having an isocenter inside the data acquisition scanner;
a bed adapted to receive the subject thereon, said bed moving movable into the subject receiving opening with the subject on the bed;
a computer that operates the bed;
one signal generator that emits a wireless signal, said one signal generator being manually positionable on the subject, outside of the subject-receiving opening, so as to be placed at a selected location that designates a part of the subject from which said image data are to be acquired;
three signal detectors mounted to said data acquisition scanner outside said subject-receiving opening, each signal detector being situated at a predetermined distance from the isocenter;
each respective signal detector detecting the wireless signal emitted by said one signal generator and each respective signal detector calculating a distance between that respective signal detector and said one signal generator, based on the detected wireless signal, and generating a detector signal that represents the calculated distance;
said computer being in communication with said three signal detectors and receiving the respective detector signals therefrom, said computer being configured to calculate, from the respective detector signals received from the three signal detectors, a three-dimensional position of the one signal generator relative to the data acquisition scanner; and
said computer being configured to operate the bed so as to move the bed with the subject thereon by an amount calculated from the predetermined respective distances of said signal detectors relative to the isocenter and the three-dimensional position of said one signal generator, so as to place said selected location at the isocenter for acquiring said image data from said selected location of the subject at the isocenter.

2. A medical imaging apparatus as claimed in claim 1 wherein said data acquisition scanner has a scanner face at which said subject-receiving opening exits said data acquisition scanner, and wherein said three signal detectors are mounted on said scanner face with all three of said signal detectors being in a same plane.

3. A medical imaging apparatus as claimed in claim 1 wherein said one signal generator is selected from the group consisting of an ultrasonic signal generator, an infrared signal generator and a laser signal generator, and wherein said three signal detectors are selected from the group consisting of ultrasonic signal detectors, infrared signal detectors, and laser signal detectors.

4. A medical imaging apparatus as claimed in claim 1 comprising a handle to which said one signal generator is mounted so as to be manually positionable relative to the subject, said handle being adapted to be held vertically in order to position said one signal generator relative to the subject at said selected location, and wherein said handle has an additional signal generator mounted thereon that moves around a vertical axis when said handle is moved around said vertical axis, said additional signal generator emitting an additional wireless signal that is detected by each of said three signal detectors, and each of said three signal detectors generating a further detector signals dependent on detection of said additional wireless signal, said further detector signals also being supplied to said computer and said computer being configured to calculate an amount of angular rotation of said handle around said vertical axis from said further detector signals.
